(19) United States
(12) Patent Application Publication
(10) Pub. No.: US 2013/0178714 A1
(43) Pub. Date: Jul. 11, 2013

(54) SURGICAL TONGUE DEPRESSOR

(71) Applicant: Philip J. Martin, Clayton, NC (US)

(72) Inventor: Philip J. Martin, Clayton, NC (US)

(21) Appl. No.: 13/782,241

(22) Filed: Mar. 1, 2013

Related U.S. Application Data
(63) Continuation-in-part of application No. PCT/US12/54337, filed on Sep. 8, 2012.
(60) Provisional application No. 61/532,203, filed on Sep. 8, 2011.

(51) Int. Cl. A61B 1/24; A61B 13/00

(52) U.S. Cl. CPC A61B 1/24; A61B 13/00
USPC 600/241; 600/240

(57) ABSTRACT

The present invention relates to a tongue depressor with a light. The present invention relates to a tongue depressor designed to be utilized when the clinician is positioned behind the head of the patient, for example, during anesthesia in surgery where insertion or removal of an airway device into the patient is necessary. The depressor comprises an anatomical bend, gripping surface for holding the depressor, a tissue grabbing surface, and a barrel end at the distal end of the grabbing and moving tissue, such as the tongue.

This application is a Continuation-in-Part of application number PCT/US12/54337 filed on Sep. 8, 2012 which claims priority to U.S. provisional application Ser. No. 61/532,203 filed on Sep. 8, 2011. All applications are included herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to a tongue depressor. In particular, the present invention relates to a tongue depressor designed to be utilized when the clinician is positioned behind the head of the patient for example during anesthesia in surgery where insertion or removal of an airway device into the patient is necessary.

2. Description of Related Art

A tongue depressor has traditionally been used to hold and retain a patient's tongue, lips, or other tissue in a particular position to aid in viewing during inspections of the mouth. The conventional tongue depressor is usually a wooden stick with a flat length and rounded at both ends. It has been satisfactorily utilized during patient office visits and in medical assistance situations for initial examinations. Sterilized versions of the tongue depressor have also been utilized in surgical situations.

In a surgical situation, the tongue depressor needs to be sterilized, and instead of being utilized solely for viewing of the interior of the mouth, is also an aid in the insertion and removal of an airway device, e.g. for surgical intubation, into the patient. This is frequently done, e.g. by an anesthesiologist, while standing behind the head of a prone patient, unlike the non-surgical use where the medical personnel using a tongue depressor usually faces the patient. The standard flat wooden depressor is still the most widely utilized tongue depressor in most hospital surgical settings. Wooden tongue depressors, however, can crack and pieces are easily displaced into the mouth, trachea, esophagus, or lung causing damages and sometimes even death. Even further, since one standard size flat depressor is utilized, problems with the curve of the oral anatomy, and hanging on to a straight blade while inserting into a curved oral cavity causes the depressor to frequently be dropped. All the standard depressors have some difficulties in providing grasp and leverage for use in inserting an airway device and as such, are fraught with problems.

Attempts at creating a better tongue depressor have led others to design expensive contraptions and a variety of other means for improving on the tongue depressor, however, because of the limitations of these devices they do not adequately correct all the problems without substantial cost and therefore, have essentially not been widely adapted and the inadequate flat wooden tongue depressor remains the device of choice in the surgical setting.

### BRIEF SUMMARY OF THE INVENTION

The present invention relates to the discovery that a depressor with a combination of elements is far superior to any depressor utilized currently in surgery especially combining plastic or metal construction; an anatomical bend; gripping surface; a tissue grabbing surface; and a tissue moving barrel end. The tissue moving barrel end is new and useful since even when prior art depressors are used the ability to move tissue is usually hampered to a great deal. The device is enhanced even further by the ability to have a light mounted on the anterior surface which eliminates the difficulties with separate lights and the ability to direct the light.

Accordingly, in one embodiment of the present invention there is disclosed a one piece surgical tongue depressor for a selected patient comprising:
a) an elongated body having a distal end and a proximal end;
b) an upper and lower surface to the elongated body;
c) a finger gripping surface on at least one of the upper and lower surface toward a proximal half of the body;
d) a tissue grabbing surface on the lower surface of the elongated body toward a distal half of the body; and
e) a barrel end at the distal end disposed toward the lower surface of the body
wherein the tongue depressor is made of a medically acceptable plastic or metal and has the option of a light on in one embodiment the anterior surface of the depressor.

### BRIEF DESCRIPTION OF THE DRAWINGS

FIG. 1 is a perspective view of the lower surface of the present invention tongue depressor.

FIG. 2 is a side view of the upper surface of the present invention tongue depressor.

FIG. 3 is a side view of the tongue depressor in use with a patient.

FIG. 4 is a view of a blade without an anatomical bend.

FIG. 5 is a perspective view of the tissue grabbing surface.

FIG. 6 is a perspective view of a straight depressor.

FIG. 7 is a top perspective view of the depressor with anterior (upper) surface light mounted thereon.

FIG. 8 is a bottom perspective view of the depressor with anterior surface light mounted thereon.

### DETAILED DESCRIPTION OF THE INVENTION

While this invention is susceptible to embodiment in many different forms, there is shown in the drawings, and will herein be described in detail, specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

The terms “about” and “essentially” mean ±10 percent.

The term “comprising” is not intended to limit inventions to only claiming the present invention with such comprising language. Any invention using the term comprising could be separated into one or more claims using “consisting” or “consisting of” claim language and is so intended.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to he considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the term “one piece” refers to the tongue depressor having been molded or shaped from a single piece of material for the body portion of the depressor. Therefore, where the device is made of a plastic, it can be molded to the desired shape. Where the material is a metal, it could be molded, stamped, or the like into the desired shape. The one piece material being either a plastic or a metal can be selected to be medically acceptable material and stiff enough and rigid enough to not break under normal use. One skilled in the art is knowledgeable enough to select proper rigidity. One advantage of plastic is, it will not splinter or shatter as wood products of the prior art would. Metal devices most likely will not break under most situations, and at worse would likely bend, thus leaving them much safer than wood devices. In addition, when a particular bend is to be placed into the body, placing that bend is much easier in a metal/plastic device than in a wooden device though in one embodiment the body is not bent, i.e. straight.

As used herein a “surgical tongue depressor” is a depressor designed to be used in a surgical setting. In general, where utilized by an anesthesiologist it is designed to be utilized with the physician positioned at the head of the patient with the patient in a prone position for surgery. In one embodiment, the depressor is sterilized for use in the surgical theater setting. In general, rather than just being utilized just to view areas in the oral cavity, it is also utilized as an aid in inserting airways during surgery for any medical procedure for that matter). While the design is for surgical purposes, the present invention tongue depressor can be utilized in any medical procedure especially where a standard wooden or flat depressor would be dangerous or less likely to work than the present invention depressor.

The tongue depressor of the present invention can be made of any medically acceptable plastic or metal usable in a surgical setting. In one embodiment, it is selected from a non-latex, biodegradable, or recyclable polymeric (plastic) material. Metals, such as surgical stainless steel, can also he utilized. One skilled in the art in view of the present disclosure could select other medically acceptable materials (medical grade) such as USP Class VI and one could combine different materials including polymers and metals. In some embodiments, the depressor is designed to be utilized once and then disposed of, however, in other embodiments it can be resterilized and reused. Disposable depressors are made of inexpensive plastic that are cheaper to use a new one than resterilizing. The plastic or metal must be of a medically acceptable material (also called medical grade metals or plastics).

Examples of such plastics include, but are not limited to, polyethylene, Polycaprolactone, PEEK, PEI, acrylics, polyvinylchlorides, polycarbonate, polypropylene, acetyls, nylons, and the like. In one embodiment, the depressor is made of a plastic that is a non-latex biodegradable or recyclable plastic. In another embodiment, the depressor is sterilized before used.

As used herein a “selected patient” refers to a patient receiving a medical procedure wherein there is a need for a surgical type tongue depressor. It can be a surgical patient, for example, placing an oral airway in an anesthetized patient but also can be utilized for Dentistry, General Medicine, Family Medicine, E.N.T. Surgery, Anesthesia, Respiratory therapy and other subspecialties.

As used herein an “elongate body” refers to the general body shape of a tongue depressor, that is a thin blade shaped body designed for insertion in the mouth for surgical or examination purposes. In general, they are only a couple to perhaps 7 or 8 inches long and less than a quarter inch in thickness. The actual length and possibly width will depend on if it is utilized with a child, a small or large adult, or the like. The width is sufficiently small to insert in the mouth from about a half inch to no more than about an inch and a half. In one embodiment, if is of even thickness except for the barrel end but can be varied thickness if desired, it is also made of a medically acceptable material (plastic or metal) of sufficient rigidity to move or hold oral cavity tissues, such as the tongue, out of the way or in place during the desired medical activity and which resists breakage (unlike wood depressors). A tongue depressor has a proximal end where it is held by the medical person using it and a distal end which is inserted into the selected patient's mouth.

Normally, a wooden tongue depressor is the same on both sides so that there is not a technical top and bottom. The present invention has an upper surface and a lower surface whose surfaces are distinguishably different. The elongated body can have a bend or be straight across the length of the depressor body (traversing the width) wherein the ends are bent toward one another on the lower surface. When there is a bend, one can make it an anatomical bend. An examination of the figures will further clarify where this bend is when utilized. In one embodiment, the anatomical bend will be an angle that is approximately adapted to the angle of the patient's oral cavity shape, i.e. an anatomical bend. Since the size and length at that bend varies as the patient changes age, size, and the like, various sizes may be necessary, i.e. a tongue depressor of the present invention designed for an adult will likely be too big and have the wrong anatomical angle for a toddler. However, those skilled in the art can fashion a device with the proper angle. Further, the bend does not need to be precisely the anatomical bend of each patient, rather smaller bends for children or small individuals and larger bends for larger adults. Approximating the bend for each is sufficient to gain the benefit of the present invention. Accordingly, one manufacturing the present invention should be able to provide 2, 3, 4, 5 to 6 sizes more or less and have fashioned enough range of sizes to accommodate most patient's oral cavities. The angle of the anatomical bend will be from 0 degrees to 90 degrees though noted, straight or any bend can be utilized. In one embodiment, the angle is 34.5 degrees. In one embodiment, the angle is 1, 2, 3, 4, 5, 6, 7, 8, 9 or 10 degrees plus or minus 34.5 degrees. In one embodiment, the blade is adjustable in its angle by making the body of a flexible material.

As used herein the “finger gripping surface” refers to a treatment of the surface of the body which increases the ability to firmly grip the tongue depressor. Usually that can be accomplished by increasing the surface area of the area to be gripped. The surface area can be increased by roughing the surface, adding grooves into the surface, adding holes to the surface, adding a series of tabs, or the like, or adding raised rubberized polymer material to the surface (even for metal depressions) in a pattern so that it has a better gripping surface. The depressor is normally grasped on the proximal half of the body and since it is gripped on both the upper and lower surface of the body in one embodiment, the gripping surface is on both sides though one side (either upper or lower) will increase the gripping. Many patterns are known by those skilled in the art for grasping including straight grooves and the like. Materials for adding gripping power to a device are well known for other purposes but have not been applied to tongue depressors. In one embodiment, the gripping surface consists of nothing being added to the tongue depressor and is some form of surface modification such as roughing, grooves, holes, or the like. In one embodiment, holes are utilized since they lighten the device and they require less material to make the device.

As used herein the “tissue grabbing surface” refers to treating, shaping, or adding to the lower surface of the elongated body toward the distal end of the elongated body in the area of that lower surface that the tongue depressor will contact the patient's tissues, e.g. where the tongue depressor would contact the tongue such that surface area is increased for the purpose of increasing the grabbing action compared to no treatment. By the “proximal end” refers to essentially the last 1% to about 25% of the proximal end of the tongue depressor lower surface. Since the upper surface is not utilized to contact tissue, there is no need for surface treatment. In one embodiment, that can be accomplished by increasing the surface area of the area to contact tissue. The surface area can be increased by roughing the surface, adding grooves into surface, or adding rubberized/polymer material to the surface in a pattern, enlarging the area such as with a raised area (e.g. semi-circular raised area or dimples) or that has a better tissue grabbing surface based on choice of materials (e.g. smooth vs. rough). In another embodiment, a bulbous projection of either the same or different material can be any shape such as circular, oval, and can be multiple projections, or the like. Many patterns can be devised by those skilled in the art for grabbing tissue in view of the present disclosure, including semi-circular grooves and the like. Materials for adding grabbing power to a device are well known for other purposes but have not been applied to tongue depressors. In one embodiment, the grabbing surface consists of nothing being added to the tongue depressor and is some form of surface modification to the device, such as roughing, grooves, or the like.

As used herein a “barrel end” on the present invention tongue depressor refers to a solid or hollow (e.g. solid or the bent portion shown n the figures) cylindrical portion to the distal end of the depressor which is disposed toward the lower surface of the elongated body across at least a portion of the width of the end of the depressor. This can be plainly seen on the embodiment of the figures which shows a barrel end disposed across the entire end of the distal end of a depressor. The barrel end is utilized to add additional leverage when using the depressor to move or hold the tongue or other tissue during use.

As used herein, the phrase “longitudinal ribs” refers to a raised area running a portion of the length of the depressor. In one embodiment, they run from the proximal end to the tissue grabbing surface. They can be positioned at the edge or middle surface, on the top surface, bottom surface c or the like. The longitudinal ribs add longitudinal stiffness and resist the devise bending in use. Other longitudinal ribs could be determined in shape and length by one skilled in the art in view of the disclosure.

In use, the tongue depressor of the present invention would be utilized to a regular depressor, however, with the optional bend and both the gripping, grabbing surfaces and the barrel end will act easier to hold and move tissue especially in surgical situations. In addition, since the device will be made of plastic or metal there is little or no possibility of the depressor breaking at a critical moment as would be the case with a wooden depressor. It is an embodiment of the present invention that wood is not a material the depressor would be made from, rather a break resistant metal or plastic.

As used herein “cross ribs” refers to raised ribs like the longitudinal ribs which are positioned diagonal to the length of the depressor. Normally at about a 35-90 degree angle (45 degree and 90 degrees perpendicular are shown in the drawings) but at any angle and height and width which adds stiffness to the length of the device along its length specifically in the middle of the length of the depressor.

As used herein the phrase “light” refers to an electric (ac or dc) operated light fixture with an appropriate bulb such as an LED or incandescent in nature. The light in one embodiment will be positioned (as shown in the figures) on the top side of the depressor and aimed toward the barrel end of the depressor. It can have batteries, an on/off switch and can have parts of it mounted anywhere so long as the light is positioned to shine in the patients mouth during use. In one embodiment, the wiring and the like is mounted inside the body yet in other embodiments it is mounted on the outside of the body for example on the top side as shown in the figures.

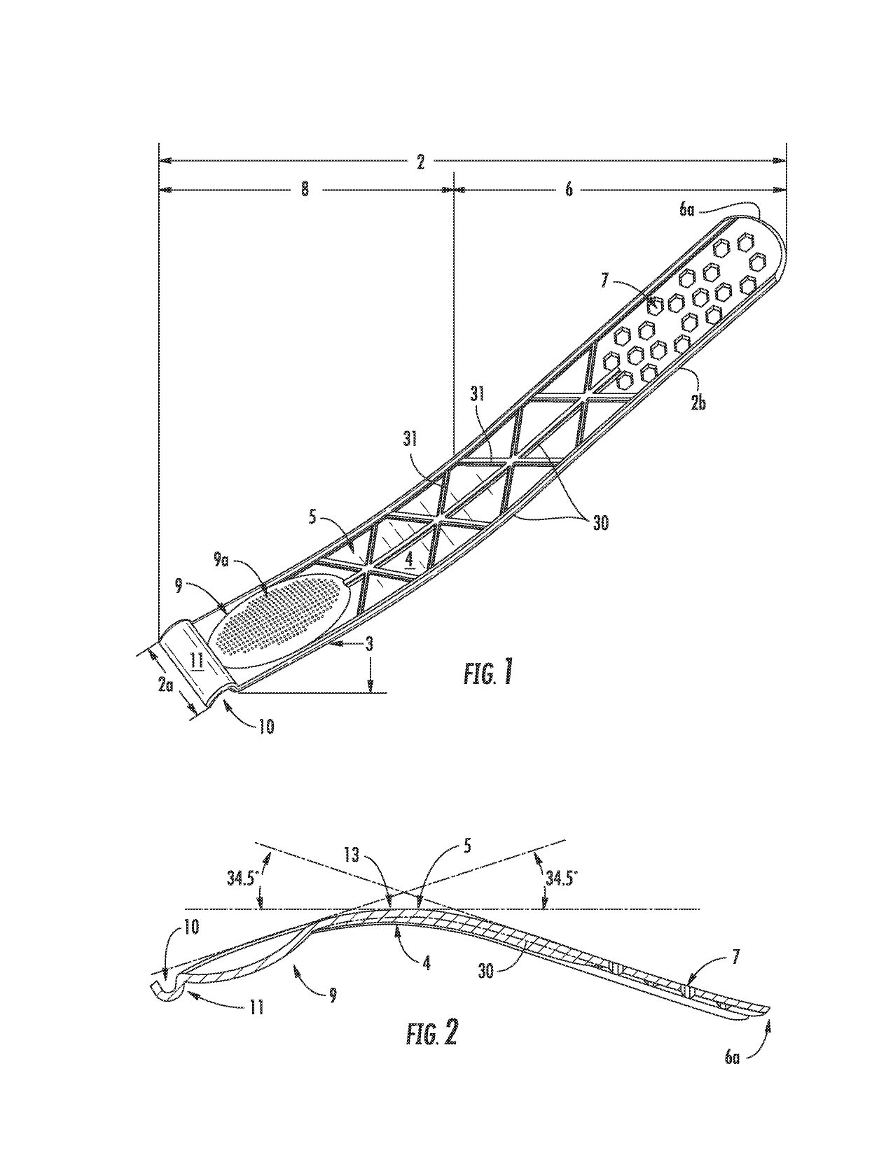

Now referring to the figures, FIG. 1 is a perspective view of a lower surface of the present invention tongue depressor. In this view, tongue depressor is shown having length 2, width 2a, and thickness 2b which is essentially the same length, width, and thickness as other tongue depressors. It has a bend of curvature 3 bending toward the lower surface 4 of body 5. In this embodiment, the angle of the bend is 34.5 degrees as shown more clearly in FIG. 2. On the proximal half 6 (having proximal end 6a) of body 5 there are a series of finger gripping hole 7 for the purpose in aiding the medical user in holding on to the depressor and for reducing cost and reducing the weight of the device. On the distal half 8 or the body 5 there is tissue grabbing, oval bulbous projection 9 positioned toward the distal end 11 of body 5. Projection 9 has raised textured surface bumps 9a for creating increased gripping. This created a depression on the opposite side of the projection 9. Also seen in this view, the distal end 11 has lower surface disposed barrel end 10. As can be seen in this view, the barrel end is not solid in this embodiment and consists of a bend at the distal end that is barrel shaped.

Also, shown in FIG. 1 are longitudinal raised ribs 30 which are positioned on either long edge and in the middle lower surface 4. Raised cross ribs 31 are shown in this view as well.

FIG. 2 is a side view of the tongue depressor showing the upper surface 13 and lower surface 4 of depressor 1 body 5. As can be seen on the lower surface 13, the gripping tabs 7 are also positioned on this side as well in this embodiment to aid in gripping the depressor 1 (though not seen). This view clearly shows how the formatting of the projection 9 creates a depression and further how the barrel is formed. The bend angle can also more clearly be seen. Also, in this view the barrel end 10 at distal end 11 can clearly be seen to be disposed away from the upper surface 13 and have its bend toward the lower surface 4.

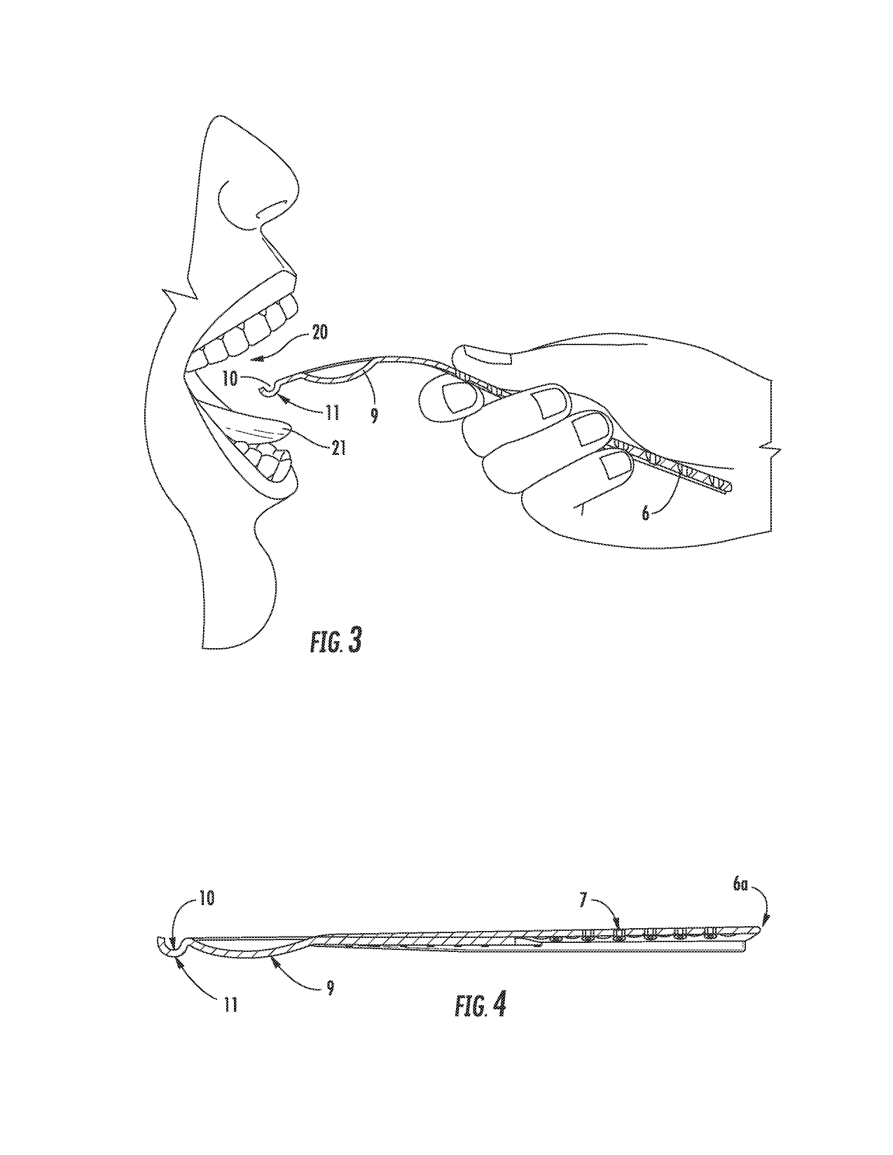

FIG. 3 is a side view of the tongue depressor of FIG. 1 in use with a patient. The barrel end 10 at distal end 11 is being placed in patient's mouth 20. Projection 9 will contact the patient's tongue 21 while being gripped on the proximal half 6.

FIG. 4 is a view of a depressor with no bend in the body 30. It is clear then, while there are several embodiments of bends in the invention, the body can be straight as well.

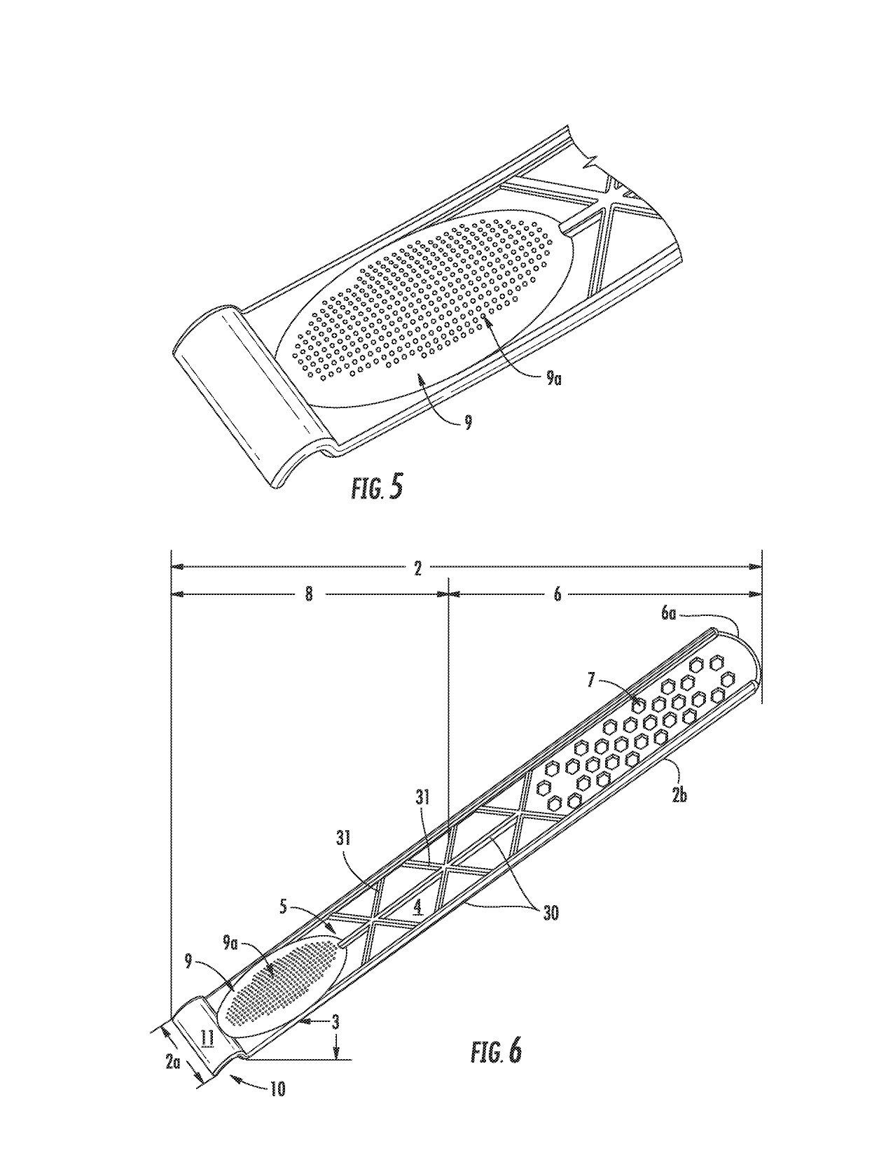

FIG. 5 is a perspective view of the tissue grabbing surface 9a, a textured surface on the projection 9.

FIG. 6 depicts a straight version of the depressor 1. The numbers on this are the same features as the curved depressor.

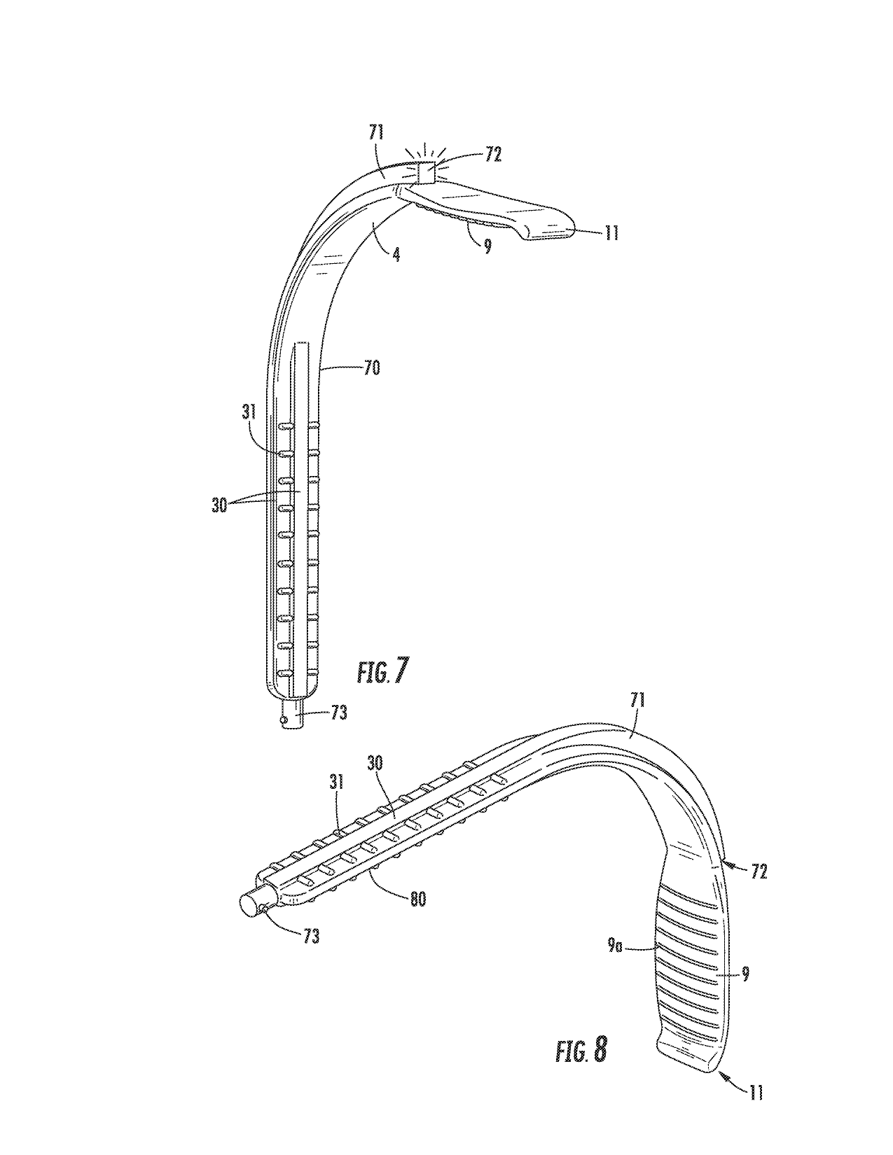

FIG. 7 is a top perspective view of a depressor of the invention with a light. In this view depressor 70 is shown with light 71. The light in this embodiment is battery driven with batteries hidden in the device by means known in the art. An on/off switch 73 operates the light and turns on bulb 72 to shine a light into patient's mouth. As can be seen by looking at FIG. 7 in combination with FIG. 3, the top mounted light 71 when turned on would shine into the patient' mouth aiding the healthcare worker in the task to be accomplished. Note that positioning it on the top surface just after anatomical bend 4 allows for the greatest degree of available light during use as long as light 71 is aimed toward the barrel end 11 of the depressor 70. It should be noted in this embodiment that the cross ribs 31 are perpendicular (90 degrees) with respect to the length of the depressor 70. In FIG. 8, the same depressor is shown in bottom perspective view. In this view, the tissue grabbing surface has horizontal ribs 9a. Also note that the barrel end 11 is not open but a solid barrel end unlike other embodiments shown.

As an example a depressor is made according to FIGS. 1 and 2 with a length of 5.85 inches, height of 0.97 inches, and a 34.5 degree bend as shown in the figures.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A one piece surgical tongue depressor for a selected patient comprising:
a) an elongated body having a distal end and a proximal end;
b) an upper and lower surface to the elongated body;
c) a finger gripping surface on at least one of the upper and lower surface toward a proximal half of the body;
d) a tissue grabbing surface on the lower surface of the elongated body toward a distal half of the body; and
e) a barrel end at the distal end disposed toward the lower surface of the body
wherein the tongue depressor is made of a medically acceptable plastic or metal.

2. The tongue depressor of claim 1 wherein the plastic is a rigid non-latex biodegradable or recyclable plastic.

3. The tongue depressor of claim 1 which is designed for single use.

4. The tongue depressor of claim 1 which is made of a medically reusable metal.

5. The tongue depressor of claim 1 wherein the finger gripping surface is a series of holes.

6. The tongue depressor of claim 1 wherein the tissue gripping surface is a bulbous projection.

7. The tongue depressor of claim 6 wherein the bulbous projection has dimples to increase the surface area and grip.

8. The tongue depressor of claim 1 which has beep sterilized for use.

9. The tongue depressor of claim 1 wherein there is a finger gripping service on both the upper and lower surface toward a proximal end.

10. The tongue depressor of claim 1 wherein there is a bend approximating the patent's oral cavity shape extending the length of the depressor wherein the ends are bent toward one another on the lower surface.

11. The tongue depressor of claim 9 wherein the bend is about 34.5 degrees.

12. The tongue depressor of claim 1 wherein there is no bend in the body.

13. The tongue depressor of claim 1 wherein there are further one or more longitudinal ribs.

14. The tongue depressor of claim 1 wherein there are one or more cross ribs.

15. The tongue depressor of claim 1 wherein there is a light mounted on the top side of the depressor positioned to shine a light toward the barrel end.

16. The tongue depressor of claim 15 wherein the light is battery operated.
